SEQUENCE LISTING (3 sequences)

SEQ ID NO: 1 - DNA, 16 bp, Artificial Sequence
  primer
ccacgctgggtgggtc

SEQ ID NO: 2 - DNA, 36 bp, Artificial Sequence
  primer
taatacgactcactatagggccacgctgggtgggtc

SEQ ID NO: 3 - DNA, 23 bp, Artificial Sequence
  primer
ctctttctcttctctctttctcc

(19) United States
(12) Patent Application Publication
(10) Pub. No.: US 2023/0093390 A1
(43) Pub. Date: Mar. 23, 2023

(54) METHOD FOR OBTAINING PROFILE OF TARGET MOLECULE POPULATION OF SAMPLE

(71) Applicant: Sung Chun KIM, Seoul (KR)

(72) Inventor: Sung Chun KIM, Seoul (KR)

(21) Appl. No.: 17/782,217

(22) PCT Filed: Dec. 2, 2020
(86) PCT No.: PCT/KR2020/017464

§ 371 Date: Jul. 28, 2022

(30) Foreign Application Priority Data
Dec. 3, 2019 (KR) 10-2019-0158887

(51) Int. Cl. C12N 15/10; C12Q 1/6869

(52) U.S. Cl. CPC C12N 15/1065; C12N 15/1048; C12Q 1/6869

(57) ABSTRACT

The present invention discloses a profiling technique for a target molecule population in a sample including an unknown target molecule, using an aptamer. In the method of the present invention, the target molecule population in the sample may be provided as an aptamer profile including an unknown target molecule, and this aptamer profile can be used to determine whether drug prescription is appropriate (i.e., anticancer drug companion diagnosis, etc.), to provide disease diagnosis information, to monitor drug treatment, to determine drug compliance, to determine the degree or absence/presence of in vitro cellular response to drug treatment, and to obtain useful information to humans for classification or identification of species, etc.

### TECHNICAL FIELD

The present invention relates to a method for obtaining a profile of a target molecule population in a sample.

### BACKGROUND

Various techniques for producing a profile, which is overall information representing an overall quantitative state of biomolecules, such as proteins constituting a sample, have been developed due to the development of physics, biochemistry, bioinformatics, etc., but there is still room for technology improvement in terms of cost, accuracy, sensitivity, and profile size.

Existing protein chips corresponding to nucleic acid chips have antibodies immobilized on a small-area microarray, and are limited in their degree of integration. The existing protein chips can profile only hundreds of molecules for known (i.e. identified) proteins unlike nucleic acid chips capable of profiling the relative expression levels of over 25,000 genes.

In particular, although biological samples contain millions of proteins, in the present time, the number of known (identified) proteins only tens of thousands. Therefore, a profiling technique for biomolecular populations of samples including unknown proteins is highly required.

Profiles of biomolecules are useful for diagnosis of various diseases, drug treatment monitoring, etc. The more diverse and mass the protein information included in the profile, the higher the usefulness of the profile.

The present invention discloses a profiling technique fora target molecule population of a sample including an unknown target molecule, using an aptamer.

### SUMMARY

### Technical Problem

The objective of the present invention is to provide a method of obtaining a profile of a target molecule population in a sample including an unknown target molecule, using an aptamer.

Other objectives and detailed objectives will be understood from a description given below.

### Technical Solution

One aspect of the present invention relates to a method of creating a profile of a target molecule population of a sample.

The method of the present invention includes: (a) treating an aptamer library tagged with a first tag that is specific to a target molecule population of a sample and is capable of binding to a first capture component, with a solid support to which the first capture component is coupled so that the first capture component and the first tag are bound to each other, thereby fixing the aptamer library to the first solid support; (b) treating an analysis target sample that is the same kind as the sample, with the first solid support on which the aptamer library is immobilized so that each target molecule of the target molecule population in the analysis target sample and each aptamer in the aptamer library form a target-aptamer complex, thereby obtaining a population of the complexes; (c) isolating the complex population in a state in which the complex population is immobilized on the first solid support by removing unbound target molecules; (d) tagging a second tag capable of binding to a second capture component to the target molecule of each complex of the isolated complex population; (e) treating the complex population tagged with the second tag, with a second solid support to which the second capture component is coupled so that the second capture component and the second tag are bound to each other and thus the complex population is immobilized on the second solid support; (f) separating an aptamer population from the complex population immobilized on the second solid support in a form in which the aptamer population is immobilized on the first solid support; and (g) generating an aptamer profile by quantifying each aptamer of the aptamer population that is still immobilized on the first solid support.

The aptamer profile thus obtained becomes the overall (that is, collective) distribution of the amount of each aptamer in the aptamer population isolated from the target molecule population of the sample, in which the amount of each aptamer is an aptamer detection value, i.e., the quantitative result of the aptamer. The amount of each aptamer is proportional to the amount of the target molecules in the sample to which the aptamer binds and represents the amount of the target molecules. Therefore, the aptamer profile is the overall (collective) distribution of the amounts of target molecules in the sample and is thus a profile for a target molecule population.

Therefore, the method of creating a profile for a target molecule population in a sample, according to the present invention, may be understood as a method of obtaining an aptamer profile for a target molecule population of a sample. As described above, this aptamer profile eventually becomes a profile fora target molecule population of a sample.

The method of the present invention is a method of crating a profile for a target molecule population of a sample. In the method, an aptamer library specific to a target molecule population of a specific sample, for example, a target molecule population of human plasma, is treated with an analysis target sample (for example, a human plasma sample) similar to the sample, and an aptamer profile for the target molecule population in the analysis target sample is generated. Such an aptamer profile or target molecule population profile can be used to provide humans with information useful for determination of suitability of drug prescription (for example, companion diagnosis of anticancer drugs, etc.), provision of disease diagnosis information, monitoring of drug treatment, determination of drug adherence, determination of degree or absence/presence of in vitro cellular response to drug treatment, classification or identification of species, etc.

In the present invention, the term ‘target molecule_refers to any molecule to be detected in a sample. Typically, the target molecules may be proteins, glycoproteins, lipoproteins, peptides, nucleic acids, carbohydrates, lipids, polysaccharides, pathogens such as viruses and bacteria, drugs, dyes, cells, and the like. Preferably, the target molecules are proteins, glycoproteins, lipoproteins, or peptide molecules.

In the present invention, such a target molecule may be an identified known molecule or an unidentified unknown molecule. In the present invention, whether the target to be detected from the sample is an identified known molecule or an unidentified unknown molecule does not matter in terms that a profile for a target molecule population is obtained as an aptamer profile. This is because, in the present invention, the detection results (quantitative results) of a certain target molecule appear as aptamer detection results (i.e., quantitative results) regardless of whether the target molecule is an identified known molecule or an unidentified unknown molecule. These aptamer quantitative results form an overall aptamer profile.

In addition, in the present invention, the term ‘target molecule population_refers to a group of two or more different molecules. As described below, an aptamer library specific to a target molecule population in a sample has a random sequence. Therefore, a single-stranded nucleic acid library having potential binding ability to various target molecules is treated, all of the target-aptamer complexes are isolated, and the nucleic acid of each of the complexes is amplified. Therefore, a number of target molecules can be collectively detected. Accordingly, the target molecule population can be understood as a target molecule group composed of at least 500 types of target molecules, preferably 1000 types or more, more preferably 1500 types or more, even more preferably 2000 types or more of target molecules.

In the present invention, the term ‘aptamer library specific to a target molecule population in a sample refers to an aggregate of aptamers capable of collectively detecting target molecules by specifically binding to the target molecule population. Therefore, When the size of the target molecule population is composed of, for example, 1000 types of target molecules, the aptamer library will also be composed of at least 1000 types of aptamers. Here, the expression ‘will be composed of at least 1000 types of aptamers_is used because, when an aptamer library is reacted with a specific target molecule, in some cases two or more specific aptamers specifically bind to the target molecule rather than a case where only one type of specific aptamer specifically binds to the target molecule. In general, the number of types of aptamers constituting the aptamer library will be greater than the number of types of target molecules constituting the target molecule population. Each aptamer of the aptamer library may preliminarily sequenced by the next generation sequencing (NGS) or by a sequence determination method such as cloning through known BAC library construction (Genome Res 2001 March; 11(3):483-496). It may be preferable to use an aptamer library that has already been sequenced as such.

In the method of the present invention, the aptamer library specific to the target molecule population in the sample in step (a) may be obtained an aptamer library preparation method conceptually described below and experimentally disclosed in the Examples below or may be obtained by an existing method.

Examples of the exiting method include a method disclosed in Korean Patent Application Publication No. 10-2019-0135456 or the corresponding International Application Publication No. WO2018/084594, which is entitled “Method for Collective Quantification of Target Protein Using Next-Generation Sequencing and Use Thereof”, and a method disclosed Korean Patent No. 10-0923048, which is titled “Nucleic Acid Chip for Generating Binding Profile between Unknown Biomolecule and Single-stranded Nucleic Acid, Method of Manufacturing Same, and Method for analyzing Unknown Biomolecule Using Same Chip”.

The method disclosed in the Korean Patent Application Publication No. 10-2019-0135456 No., etc. obtains by (i) preparing a single-stranded nucleic acid library having a variety of different sequences (i.e., random sequences), thereby having potential binding ability to various target molecules, (ii) reacting the single-stranded nucleic acid library with a target molecule population in a sample to induce specific binding between each single-stranded nucleic acid and each target molecule to form a complex population, (iii) isolating the complex population by excluding unbound single-stranded nucleic acids, and (iv) amplifying the single-stranded nucleic acids of the complex population.

In the method for preparing an aptamer library specific to the target molecule population of the sample, when the target molecule population is a target protein group, the sample is processed on a nitrocellulose membrane disc capable of immobilizing proteins so that the target proteins can be collectively immobilized, and a single-stranded nucleic acid library is processed thereon.

In the process of preparing the aptamer library, the single-stranded nucleic acid library having different sequences in step (i) generally refers to a single-stranded RNA or DAN oligonucleotide nucleic acid pool, and the oligonucleotide is composed of a 5′ end conserved region consisting of a known sequence, a 3′ end conserved region, and a variable region consisting of a random sequence between the 5 ̆ end conserved region and the 3 ̆ end conserved region. The conserved region composed of this known sequence may include a sequence to which forward/reverse primers bind, a promoter sequence for RNA polymerase, a restriction enzyme recognition sequence for manipulation such as cloning, and the like. Since the variable region consisting of the random sequence usually consists of 40 to 60 nucleotides, the total length of the oligonucleotide including the 5′ end region and the 3′ end region usually corresponds to 60 to 150 nucleotides. The synthesis of such oligonucleotides is well known in the art, and examples of the synthesis method include a solid-phase oligonucleotide synthesis technique and a solution-phase synthesis technique such as a triester synthesis method. For the details, refer to Literature [Nucl. Acid Res. 14:5399-5467, 1986] Literature [Vet Lett 27:5575-5578, 1986], Literature [Nucl. Acid Res. 4:2557, 1977], Literature [Lett, 28:2449, 1978], etc. In addition, an automated nucleic acid synthesizing apparatus available on the market may be used. When using such an apparatus, a single-stranded nucleic acid library having a sufficient size, including 10¹⁴-10¹⁶ oligonucleotides can be obtained. In addition, when a single-stranded RNA library is used as the single-stranded nucleic acid library, the RNA library can be obtained by transcribing the DNA library with an RNA polymerase such as T3, T4, or T7.

In addition, in the preparation of the aptamer library, the removal of the unbound single-stranded nucleic acid for the isolation of the complex population in step (iii) is achieved by performing washing one or more times with an appropriate washing buffer, using an appropriate known method. After removing the unbound single-stranded nucleic acid and “selectively_isolating the complex population, it is possible to construct an aptamer library specific to the target molecule population of the sample by ‘amplifying_only the single-stranded nucleic acids of the complex population. The aptamer library obtained by performing such selection and amplification only once can be used as it is in step (a) of the method of the present invention. However, by repeating the selection and amplification process two or more times, that is, by reacting again the aptamer library obtained by amplifying only the nucleic acids with the target molecule population of the sample to form a complex population again, isolating the complex population again, and amplifying only the aptamer of the complex again, it is possible to use an aptamer library having increased specific binding ability to a target molecule population in an analysis target sample. However, it is preferable that more diverse target molecules can be collectively detected and analyzed by reflecting the target molecules of the analysis target sample in a variety of ways, in terms of the usefulness of the aptamer library obtained for the target molecule population. Therefore, instead of constructing an aptamer library by performing the selection and the amplification once, it is preferable to construct an aptamer library by performing selection and amplification one time to obtain an aptamer library and performing washing two or more times using various washing buffers. As the washing solution, a washing solution widely used in the art may be purchased for use, or a washing solution may be appropriately prepared. Examples of the washing solution generally include a surfactant and/or a salt (chaotropic salt). Examples of the surfactant used may include SDS, Tween 20, Tween 30, Tween 40, Tween 60, Tween 80, Triton X-405, Triton X-100, Tetronic 908, Cholesterol PEG 900, Polyoxyethylene Ether W-1, Span 20, Span 40, Span 85, mixtures thereof. Examples of the salt used may include acetate, lactate, citrate, phosphate, nitrate, sulfate, perchlorate, and chloride of lithium, sodium, potassium, magnesium, and ammonium, and mixtures thereof (for example, SSC, SSPE, etc.). In particular, as the washing solution, TBST solution (10 mM Tris-Cl, pH 8.0, 150 mM NaCl, 0.05% Tween 20), PBST solution (PBS, pH 7.0, 0.05% Tween 20), SS PE solution (0.2M phosphate buffer, 2.98 M NaCl, 20 mM E DTA, pH7.4) containing Tween 20 and/or Tween, SB18 solution (40 mM HEPES, pH 7.5, 101 mM NaCl, 5 mM KCl, 5 mM MgCl₂, and 0.05% by volume (v/v) of Tween 20), SB17 solution (SB18 solution with 1 mM trisodium EDTA added), SB17T solution (40 mM HEPES, pH 7.5, 102 mM NaCl, 5 mM KCl, 5 mM MgCl₂, 1 mM EDTA, and 0.05% of Tween 20), etc. may be used. IN addition, 1 to 600 mM EDTA solution or the like can also be used.

Before washing to remove the unbound single-stranded nucleic acid, in order to improve specific binding to the target molecule to prevent the single-stranded nucleic acid from non-specifically binding to a non-target molecule, a competitor molecule that can bind to an arbitrary molecule in the sample to form a non-specific complex may be added when or after forming a complex by reacting the single-stranded nucleic acid library in step (ii) with the target molecule population in the sample to form a complex or a complex population. When such a competitor molecule is added, non-specific binding of the single-stranded nucleic acid to the non-target molecule is prevented, so that the specific binding (or specific binding ability) of the single-stranded nucleic acid to the target molecule can be improved. Examples of the competitor molecule include polyanionic molecule: such as oligonucleotides, heparin, herring sperm DNA, single-stranded salmon sperm DNA; polydextran such as dextran sulfate; abasic phosphodiester polymers; dNTPs; pyrophosphate, etc. The competitor molecules may be polycation molecules such as spermine, spermidine, polylysine, and polyarginine, or amino acids such as arginine or lysine. It is preferable that the concentration of the competitor molecules is higher than an expected concentration of target molecules or the concentration of single-stranded nucleic acids, in terms of improving the specific binding of the single-stranded nucleic acid to the target molecule. In addition, these competitor molecules may be added to the washing buffer during the washing step.

In this description, unless otherwise defined, the term ‘aptamer_means a nucleic acid ligand having specific affinity to a specific target molecule, and such an aptamer is a candidate nucleic acid having a potential binding ability to a specific target molecule and is obtained through the selection and amplification processes as described above. Here, when the aptamer has specific affinity to a target molecule, the specific affinity refers to a case where the aptamer specifically binds only to the target molecule to form a complex and substantially does not bind to the other molecules to form complexes. Since it is satisfactory that the complex is not substantially formed, it does not mean that the case where the complex is formed by non-specific binding is excluded because the complex can be removed by washing or the like.

Also, in the present invention, an aptamer having specific affinity to a target molecule is a non-naturally occurring nucleic acid ligand obtained through artificial preparation, selection, and amplification. Such an aptamer is generally single-stranded DNA or single-stranded RNA, and examples of the aptamer may include a modified nucleotide as well as a natural nucleotide as long as the nucleotide has the ability to bind to a target molecule. The modified nucleotides may be modified from sugar such as ribose or deoxyribose, from phosphate, and/or from base thereof. Nucleotides obtained by modifying sugars, phosphates, and/or bases, and preparation methods thereof are known in the art. For example, the sugar-modified nucleotides may be obtained by modifying the 2′ position of sugar with a halogen group (especially fluorine (F)), an aliphatic group, an ether group, or an amine group. E specially, the sugar-modified nucleotides may be obtained by modifying the 2 ̆position of sugar with OMe, O-alkyl, O-allyl, S-alkyl, S-allyl, or halogen. Alternatively, the sugar-modified nucleotides may be ones in which ribose or deoxyribose is substituted with sugar analogs such as -anomeric sugars, substituted with epimeric sugars such as arabinoses, zyloses, or lyxoses, or substituted with pyranose sugars or furanose sugars. The phosphate may be modified into P(O)S(thioate), P(S)S(dithioate), P(O)NR2(amidate), P(O)R, P(O)OR′, CO, or formacetal (CH₂). Herein, R or R′ is H or substituted or unsubstituted alkyl. When modified from phosphate, the linking group may be —O—, —N—, —S—, or —C— and the adjacent nucleotides bind to each other via this linking group. The modified nucleotides, the single-stranded nucleic acids including such modified nucleotides, etc. are disclosed in many literatures known in the art, such as [S proat et al, Nucl Acid Res 19:733-738(1991)], [Cotten, et al, Nucl Acid Res 19:2629-2635(1991)], and [Hobbs, et al, Biochemistry 12:5138-5145(1973)]. Therefore, for the details thereof, reference can be made to these literatures. All literatures cited herein, including all of these literatures, are considered part of this specification.

In addition, in the present invention, the sample may be any mixture or solution that contains or is suspected to contain at least one or a considerable number of target molecules to be detected so that detection of the target molecules is required. The sample may be not only a biological sample obtained from a human or animal, but also a processed sample in which the concentration of target molecules to be detected is increased by processing the biological sample. Alternatively, the sample also may be water, food, industrial wastewater, or the like that needs to be tested for environmental pollutants and toxic factors. These samples may contain appropriate diluents and buffers. When it is required to detect the presence of bacteria or viruses, the sample may be a bacterial culture, a bacterial lysate, or a virus culture containing a medium or components of a medium.

In the method of the present invention, preferably the sample may be a biological sample obtained from a human body or an animal, or a processed sample thereof. The biological sample may be obtained from a human body or an animal body that needs to the tested because the human or animal body is suspected to contain target molecules to be detected. For example, the sample may be blood, urine, saliva, semen, amniotic fluid, lymph fluid, sputum, and tissue, or the like. The processed sample may be, for example, a sample obtained by processing a biological sample such as plasma, or serum with a protein extraction kit to increase the concentration of proteins. Alternatively, the processed sample may be a tissue extract cells obtained from a tissue, a cell lysate, a cell culture, or the like. In addition, the processed sample may be a sample obtained by removing proteins present in a large amount in the original biological sample and unlikely being usable as target proteins (i.e., unlikely being used as a biomarker for a specific disease, etc.) from the original biological sample. For example, a blood sample contains a few types of proteins, and the few types of proteins account for 99.9% of the total amount of proteins in the sample. Such a dominant protein in amount in the sample is not so useful as a target protein (Mol Cell Prot (2006) 5(10):1727-1744). When a processed sample from which a protein present in a large amount in an original biological sample is removed is used, the detection sensitivity of a useful target protein can be improved. In the case of a blood sample of a mammal such as a human, proteins present in such a large amount include, for example, albumin, IgG, IgA, transferrin, fibrinogen, and the like. These proteins present in substantial amounts in a sample can be removed by using an appropriate method known in the art (for example, immuno-affinity depletion) or using an appropriate commercially available kit (Agilent Technologies' Multiple Affinity Removal System, etc.).

In the present invention, the expression that the sample to be analyzed in step (b) is ‘the same kind as_the sample in step (a) means that when the sample to be analyzed in step (b) is a sample such as serum derived from a specific person, the sample in step (a) refers to a sample such as serum derived from the same origin (the same person) or from a different origin (a different person). In addition, for example, the term ‘sample of same kind_means that that when a certain sample in step (a) is an E. coli lysate belonging to a specific strain, the sample to be analyzed in step (b) is an E. coli lysate belonging to the same strain or an E. coli lysate belonging to another strain. In general, in the method of the present invention, the aptamer library specific to the target molecule population of the sample in step (a) will be used in a prepared state (that is, previously prepared and identified in its sequence as necessary) in the present invention.

In the present invention, the step of isolating the complex population immobilized on the first solid support by removing the unbound target molecules in step (c) is performed using an arbitrary method known in the art depending on the nature of the first solid support used. For example, when the first solid support is a magnetic bead, the first solid support on which the complex population is immobilized is collected using a magnet, and the remaining unbound target molecules are removed. Alternatively, the first solid support is centrifuged to remove an upper layer containing the unbound target molecules and to collect a lower layer containing precipitate.

In the present invention, the capture component is an arbitrary molecule capable of covalently or non-covalently binding to a tag. The capture component performs a function of immobilizing a single-stranded nucleic acid or an aptamer to which a tag is bound, on a solid support. The capture component may be streptavidin or an analog thereof when the tag is biotin or a biotin analog. Examples of the biotin analog include desthiobiotin and iminobiotin, and examples of the analog of streptavidin include avidin, traptavidin, neutravidin (Thermo Fisher Scientific Inc., USA), and the like.

Because the tag and the capture component specifically bind to each other, they can be used interchangeably. For example, streptavidin or an analog thereof may be used as the tag, and biotin or an analog thereof may be used as the capture component.

A tag is covalently or non-covalently bound to a single-stranded nucleic acid or an aptamer. Regarding a method of binding a tag to a single-stranded DNA, RNA nucleic acid or an aptamer, various methods are known in the art. For examples, there are many literatures disclosing the methods: [Nucleic Acids Res 1987 J un 11; 15(11): 4513-4534], [RNA 2014 March; 20(3): 421-427], [Methods Mol Biol 2009; 498:185-196], etc. For the methods, various kits are commercially available (from Thermo F is her Scientific Inc., and APExBIO Technology LLC).

In particular, in the present invention, when the target molecule is a protein and the second tag is biotin, the attachment of the second tag can be made by using NHS-PEG 4-biotin, which can covalently bind the biotin to the protein, particularly to a lysine residue of the protein.

In the method of the present invention, the solid support is an arbitrary insoluble support having a surface to which a capture molecule can be directly bound via a covalent or non-covalent bond (the capture component is directly bound to the solid support) or to which a capture molecule can be indirectly bound (the capture component is attached to the solid support via another component).

The solid support may be glass, silicone, synthetic resin (for example, polycarbonate, nylon, nitrocellulose, polystyrene, polyurethane, polyacrylamide, polyacrylate, polyethylene, polypropylene, poly(4-methylbutene), polystyrene, polymethacrylate, polytetrafluoroethylene), graphite, agarose, germanium, gold, silver, or the like.

The solid support may be prepared in the form of a strip, a plate, a disk, a rod, a membrane (for example, a polyethylene, polypropylene, or polyamide membrane), particles, beads, a tube, a well plate, or a wafer.

The solid support may be porous, non-porous, magnetic, paramagnetic, nonmagnetic, polydisperse, monodisperse, hydrophilic, hydrophobic (hydrophobic), etc.

The solid support may have a functional group such as a carboxy group, an epoxy group, a tosyl group, an amino group, or a siloxane group to enable binding to a biomolecule such as a protein or a nucleic acid or to a capture molecule. Methods for introducing such a functional group to a solid support are known in the art, for example, U.S. Pat. Nos. 6,027,945, 6,673,631, 7,183,002, Japanese Patent No. 3,253,638, Japanese Patent Application Publication No. 2001-136970, Korean Patent Application Publication No. 2009-0088299, etc.

In the present invention, the solid support, particularly the first solid support, may be preferably magnetic particles (or magnetic beads) that can be easily isolated by a magnet. As disclosed in U.S. Pat. Nos. 5,665,554, 6,027,945, 7,078,224, Korean Patent Application Publication No. 2006-0061494, Korean Patent No. 0541282, etc., magnetic particles have been widely used for isolation of proteins, nucleic acids, cells, bacteria, etc. and have sizes in a range of from micrometers to nanometers (several micrometers, or tens to hundreds of nanometers). Magnetic particles that have been typically used for isolation of biomaterials are iron oxide magnetic particles. As such iron oxide magnetic particles, magnetite (Fe₃O₄), maghemite (Fe₂O₃), hematite (Fe₂O₃), etc. are used. In order for magnetic particles to be used for the isolation of biomolecules such as proteins and nucleic acids, magnetic particles are modified with non-magnetic materials such as silica, polymer, gold, or silver to prevent interaction between and aggregation of the magnetic particles, and are added with a functional group capable of binding to biomolecules, etc., for example, a carboxyl group, an epoxy group, a tosyl group, an amino group, and a siloxane group. In the market materials into which biotin, streptavidin, or the like is introduced through these functional groups have already been commercially available.

In the present invention, when biotin is used as the tag, it is preferable to select or purchase an appropriate solid support; such as magnetic particles, into which streptavidin is introduced, from among commercially available solid supports. Examples of the solid support include Dynabeads M-280 streptavidin, Dynabeads MyOne streptavidin, Dynabeads M-270 streptavidin (Invitrogen), Streptavidin Agarose Resin (Pierce), Streptavidin Ultralink Resin, MagnaBind Streptavidin Beads (Thermo Scientific Inc.), Biomag Streptavidin, ProMag Streptavidin, Silica Streptavidin (Bangs Laboratories), Streptavidin Sepharose High Performance (GE Healthcare), Streptavidin Polystyrene Microspheres (Microspheres-Nanospheres), and Streptavidin Coated Polystyrene Particles (Spherotech), and the like.

In the present invention, it is preferable that the second solid support differs in characteristics from the first solid support. This is because it is easy to separate the first solid support to which the complex population or the aptamer population is fixed in the case where the second solid support differs in characteristics from the first solid support when isolating the complex population in step (c) or when isolating the aptamer population in step (g). Accordingly, when the first solid support is a magnetic support; the second solid support may be a non-magnetic support.

In the present invention, when forming the complex of the target molecule and the aptamer in step (b), the competing molecules described above and the sample may be treated together with the first solid support to enhance the specific binding between the target molecules and the aptamers. These competing molecules may be added before the unbound target molecules are removed by treatment with the washing buffer as described above in step (c) after the complex formation. Alternatively, the competing molecules may be used in a state of being mixed with the washing buffer when removing the unbound target molecules through the washing process in step (c). In addition, when the complex population fixed to the second solid support is washed with the washing buffer as described above to remove the unreacted components (for example, unreacted second tag not attached to the target molecule, aptamer immobilized on the first solid support and not bound to the target molecule, etc.) after the complex population is fixed to the second support in step (e), the competing molecule may be added together with the washing buffer (i.e., the competitor molecule may be used in a state of being added to the washing buffer), or the competitor molecule may be added to the complex population before the washing. The treatment with such a competitor molecule may prevent non-specific binding between the aptamer and the non-target molecule and promote the specific binding between the aptamer and the target molecule.

In the present invention, the step of isolating the aptamer population from the complex population immobilized on the second solid support in step (f) in such a form that the aptamer population is fixed to the first solid support, is a step of isolating the aptamer in a state of being bound to the first solid support, by breaking the non-covalent bond between the target molecule and the aptamer in the complex population bound in a sequence of the second solid support-the target molecule-the aptamer-the first solid support. This step may be accomplished using any method known in the art capable of breaking the non-covalent bond between the target molecule and the aptamer. Examples of the method include heating, treatment with a chaotropic salt in the washing process, pH change to strongly acidity or strongly basicity, treatment with a surfactant in the washing process, and combinations thereof.

In the present invention, after the aptamer population is isolated in a form immobilized on the first solid support; an aptamer profile is generated from the aptamer population in step (g). The aptamer profile can be generated by quantifying each aptamer in the aptamer population and comprehensively dealing with the resulting data.

Methods for quantifying aptamers are also known in the art. As such methods, for example, a method using next generation sequencing (NGS), a method using a microarray, a multiplex real time PCR method, etc. are exemplary methods.

When quantifying the aptamers using the NGS method, Roches̆ 454 platform, Illuminas̆ HiSeq platform, Life Technologys̆ Ion PGM platform, Pacific BioSciences̆ TPacBio platform, etc. can be used, and quantitative results of the aptamers can be obtained according to the protocol of the device. This NGS technology spatially separates the DNA library obtained by fragmenting genomic DNA into individual fragments on a substrate or emulsion (bead), amplifies by PCR to form clones of each fragment and performs a sequencing reaction in a massively parallel manner for hundreds of thousands to billions of clones, thereby simultaneously reading the sequences of each clone. In this sequencing reaction, a single DNA fragment of each clone is used as a template to attach mononucleotides one by one by the polymerase chain reaction (PC R), and the signal generated at that time is physically and chemically detected. Reads, which are sequence information obtained for each fragment, can be compared with a preliminarily prepared reference sequence, counted by bioinformatics techniques, and quantified.

In the method using a microarray, a probe complementary to an aptamer is fixed to one or more spots on a support, the aptamer is processed on the spot to induce hybridization, and the degree of hybridization is detected with a signal generating material.

The support that can be used may be made of a material such as glass, silicone, synthetic resin, etc. The support may be manufactured in the form of a flat substrate such as a slide or a membrane, a cross-sectional form of a fiber bundle, or a bead or particle form.

The signal generating material may be a radioactive isotope, a fluorescent material, a chemiluminescent material, an enzyme, etc. Specifically, for example, the signal generating material may be cyanine fluorescent dye (Cy2, Cy3, Cy5), Alexa Fluor 350, 430, etc., fluorescein, bodipy, Texas red, Fluorescein Isothiocyanate (FITC), rhodamine, horseradish peroxidase, biotin, SYTO (SYTO-11, etc.), ethidium bromide, ethidium homodimer-1, ethidium homodimer-2, ethidium derivative, acridine, acridine orange, acridine derivative, ethidium-acridine heterodimer, ethidium monoazide, propidium iodide, 7-aminoactinomycin D, POPO-1, TOTO-3, or the like. The signal generating material may be labeled and used in the preparation of the aptamer, or may be added and used after hybridization in the case of an intercalator. A labeling method for preparing an aptamer is known in the art. For example, reference may be made to the method described in [Nucleic Acid Microarray and the Latest PCR Method] (published by Shujunsha, Japan).

The degree of hybridization can be detected with an image scanner of a biochip such as a fluorescent image scanner, and the degree of hybridization can be quantitatively calculated by software that can process image signals statistically and numerically.

The multiplex real-time PCR method uses the 5′-3′ exonuclease activity of a DNA synthetase to synthesize several template sequences (herein, aptamers) to be amplified. During the synthesis, when digesting probes (for example, TaqMan probe) complementary to respective template sequences, different signals generated by the respective template sequences may be simultaneously detected. Alternatively, different signals generated by respective templates may be simultaneously detected with insertable fluorescent substances that emit different signals during the replication process. The signal increases with the degree of replication, and the amount of replicated PCR product depends not only on the starting concentration of the aptamers but also on the number of replication cycles performed.

The multiplex real-time PCR method includes a multiplex real-time multiplex RT-PCR method using RNA as an initial template as well as the multiplex real-time PCR method using DNA as a template.

In another aspect, the present invention relates to a method for distinguishing two or more analysis target samples from each other.

The method includes (a) treating an aptamer library tagged with a first tag that is specific to a target molecule population in a sample and is capable of binding to a first capture component, with a solid support to which the first capture component is coupled so that the first capture component and the first tag are bound to each other, thereby fixing the aptamer library to the first solid support; (b) treating an analysis target sample that is the same kind as the sample, with the first solid support on which the aptamer library is immobilized so that each target molecule of the target molecule population in the analysis target sample and each aptamer of the aptamer library form a target-aptamer complex, thereby obtaining a complex population; (c) isolating the complex population in a state in which the complex population is immobilized on the first solid support by excluding unbound target molecules; (d) attaching a second tag capable of binding to a second capture component to the target molecule of each complex of the isolated complex population; (e) treating the complex population tagged with the second tag, with a second solid support to which the second capture component is coupled so that the second capture component and the second tag are bound and thus the complex population is immobilized on the second solid support; (f) isolating an aptamer population from the complex population immobilized on the second solid support in a form in which the aptamer population is immobilized on the first solid support; (g) generating an aptamer profile by quantifying each aptamer of the isolated aptamer population that is still immobilized on the first solid support; (h) performing steps (a) to (g) in the same manner on one or more analysis target samples different from the analysis target sample, thereby generating an aptamer profile for a target molecule population of each of the one or more different analysis target samples; and (i) comparing the aptamer profile generated in step (g) with the aptamer profile generated in step (h) to determine one or more aptamers having a difference in aptamer quantitative results.

In the method of the present invention, an aptamer profile is obtained in step (h) for another analysis target sample, and this aptamer profile is compared with the aptamer profile obtained in step (g). Thus, this method can be used to distinguish two samples from each other. In the method of the present invention, aptamer profiles are generated for two different analysis target samples in step (h), and the obtained two aptamer profiles are compared with the aptamer profile obtained in step (g). In this case, the method can be used to distinguish three samples from each other. In this way, the method of the present invention can be used to distinguish two or more samples.

In particular, the method of the present invention may further include a step of generating an averaged aptamer profile by averaging two or more aptamer profiles generated in step (h) for two or more analysis target samples, respectively. In this case, step (i) of comparing the averaged aptamer profile with the aptamer profile obtained in step (g) may be used for sample classification.

The averaged aptamer profile (trained aptamer profile) can be obtained by calculating an average value of the quantitative results of the same aptamer in the two or more aptamer profiles (for example, aptamer a ̆in the aptamer profile of sample A and the same aptamer a ̆in the aptamer profile of sample B), and deriving an overall distribution of the average values for the respective aptamers.

The averaged aptamer profile may be of two types. For example, one is obtained from two or more aptamer profiles of a diseased person and the other is obtained from two or more aptamer profiles of a normal person.

In the case where one averaged aptamer profile is obtained for hundreds of samples or thousands of samples from a diseased person, and another averaged aptamer profile is obtained for hundreds or thousands of samples from a normal person, when classifying a subject as a diseased person or a normal person by comparing the aptamer profile of the analysis target sample of the subject with the two types of averaged aptamer profiles described above, the accuracy can be increased.

In the method, the same aptamer library is preferably used for two or more types of analysis target samples as the aptamer library in step (a). Here, the same aptamer library refers to an aptamer library having the same aptamer composition. When the same aptamer library is used, since the aptamer for each target protein is the same (i.e., the aptamer having the same sequence), the binding force between the target protein and the aptamer is the same. Therefore, there is no difference in quantitative results depending on the binding force, and the quantitative results thus obtained can be used more usefully in classifying two or more samples.

In addition, in the method of the present invention, after step (i), a step described below may be further performed. In the additional step, with the use of one or more aptamers determined to be different in the quantitative results obtained in step (i), a target molecule to which the aptamer specifically binds is isolated, and then the isolated target molecule is identified using a method (for example, MALDI-TOF when the target molecule is a protein) known in the art. The identification of such target molecules has the effect of providing useful candidates of disease-specific biomarkers.

In addition, in the present invention, a step of repeating steps (a) to (i), using one or more aptamers that have been ‘previously_determined to be different in the quantitative results obtained in step (i) instead of the aptamer library in step (a). In this case, since the one or more aptamers are aptamers that have already been confirmed to have a difference in quantitative results between the two or more analysis target samples, the effect of more easily distinguishing the two or more analysis target samples from each other can be obtained. The one or more aptamers may be in a state in which the sequences thereof have already been determined and prepared before performing steps (a) to (i).

In the method of the present invention, the target molecule is preferably a protein, a glycoprotein, a lipoprotein, or a peptide.

Also, in the method of the present invention, the target molecule population may include an unknown target molecule.

In addition, in the method of the present invention, the aptamer library specific to the target molecule population in the sample may obtained preferably by: (i) preparing a single-stranded nucleic acid library having a variety of different random sequences and thus having potential binding ability to various target molecules; (ii) reacting the single-stranded nucleic acid library with the target molecule population in the sample to induce specific binding between each single-stranded nucleic acid and each target molecule to form a complex population; (iii) isolating the complex population by excluding unbound single-stranded nucleic acids; and (iv) amplifying the single-stranded nucleic acids of the complex population. In this case, steps (i) to (iv) are performed once, the excluding of the unbound single-stranded nucleic acids is performed through a washing step, the washing step is repeated two or more times, and the washing step is performed using a washing buffer containing a surfactant, a salt, a competitor molecule, or a mixture thereof.

In addition, in the method of the present invention, the aptamer may be single-stranded RNA or single-stranded DNA, and the single-stranded RNA or single-stranded DNA may include nucleotides modified from sugars, phosphates, or bases.

In addition, in the method of the present invention, preferably, the sample may be a biological sample such as blood, or a processed sample thereof, such as plasma, serum, or the like.

In the method of the present invention, the first tag and the second tag may be biotin or an analog thereof, and the first capture component and the second capture component may be streptavidin or an analog thereof.

In the method of the present invention, the first solid support may be a magnetic bead, and the second solid support may be a non-magnetic support.

In the method of the present invention, when the target molecule and the aptamer form the complex in step (b), the competitor molecule and the sample are processed together on the first solid support to improve the specific binding between the target molecule and the aptamer.

In the method of the present invention, the isolating of the aptamer population from the complex population immobilized on the second solid support in step (f) in a form in which the aptamer population is immobilized on the first solid support; comprises: heating, treatment with a chaotropic salt, inducing a change of pH to strong acidity or strong basicity, treatment with a surfactant or a combination thereof.

In addition, in the method of the present invention, the quantification of the aptamer in step (g) may be preferably performed by next-generation sequencing, microarray, or multiplex real-time PCR.

For other technical matters that are not specifically described for the sample classification method of the present invention, the description regarding the method of generating a profile of a target molecule population in a sample is valid as it is, and the relevant part can be referred to.

In another aspect the present invention relates to a method of preparing an aptamer library specific to a target molecule population in a sample.

A method of preparing an aptamer library specific to a target molecule in a sample, the method including: (a) preparing a single-stranded nucleic acid library having potential binding ability to various proteins by having a random sequence and by being tagged with a first tag capable of binding to a first capture component; (b) treating the single-stranded nucleic acid library tagged with the first tag, with a first solid support to which the first capture component is coupled so that the first capture component and the first tag bind to each other, thereby immobilizing the single-stranded nucleic acid on the first solid support; (c) treating the sample with the first solid support on which the single-stranded nucleic acid library is immobilized so that each target molecule of a target molecule population in the sample reacts with each single-stranded nucleic acid of the single-stranded nucleic acid library, thereby producing a complex population of the target molecules and the single-stranded nucleic acids; (d) isolating the complex population in a state of being immobilized on the first solid support by removing unbound target molecules; (e) attaching a second tag capable of binding to a second capture component to the target molecule of each complex in the isolated complex population; (f) treating the complex population tagged with the second tag, with a second solid support to which the second capture component is coupled so that the second capture component and the second tag are bound, thereby immobilizing the complex population on the second solid support; (g) eluting the single-stranded nucleic acid population bound to the target molecules in the complex population immobilized on the second solid support in a form in which the single-stranded nucleic acids are immobilized on the first solid support; and (h) generating an aptamer profile, which is a single-stranded nucleic acid population specific to the target molecules in the sample, from the single-stranded nucleic acid population immobilized on the first solid support.

In addition, in the method of the present invention to prepare an aptamer library specific to a target molecule population in the sample, the sequence of each aptamer constituting the library is determined by cloning through BAC library construction (Genome Res 2001 March; 11(3):483-496) or according to a method known in the art, such as next generation sequencing.

In the method of the present invention, after step (h), a step of determining the sequence of each aptamer in the aptamer library is preferably performed.

In the method of the present invention, the target molecule may be preferably a protein, glycoprotein, lipoprotein, or peptide.

In the method of the present invention, the target molecule population may include a target molecule that is not identified.

In the method of the present invention, the single-stranded nucleic acid library in step (a) includes a 5′ conserved region, a 3′ conserved region, and a random sequence disposed between the 5 ̆conserved region and the 3 ̆conserved region.

In the method of the present invention, steps (a) to (h) may be performed once, the removing of the unbound single-stranded nucleic acids of step (d) may be performed by washing the complex population formed in step (c), the washing may be repeated two or more times, and the washing may be performed using a washing buffer containing a surfactant a salt a competitor molecule, or a mixture thereof.

In the method of the present invention, steps (a) to (h) may be performed once, the complex population immobilized on the second solid support may be washed after step (f), the washing may be repeated two or more times, and the washing may be performed using a washing buffer containing a surfactant, a salt, a competitor molecule, or a mixture thereof. This washing step is to remove the complex caused by non-specific binding between the target molecule and the single-stranded nucleic acid, thereby isolating only the complex caused by the specific binding between the target molecule and the single-stranded nucleic acid.

In addition, in the method of the present invention, the aptamer may be preferably single-stranded RNA or single-stranded DNA.

In addition, in the method of the present invention, the single-stranded RNA or single-stranded DNA may include nucleotides modified from sugar, phosphate, or base.

In addition, in the method of the present invention, preferably, the sample may be a biological sample or a processed sample thereof.

In the method of the present invention, the first tag and the second tag may be biotin or an analog thereof, and the first capture component and the second capture component may be streptavidin or an analog thereof.

In the method of the present invention, the first solid support may be a magnetic bead, and the second solid support may be a non-magnetic support.

In the method of the present invention, when the target-aptamer complex is obtained in step (c), the competitor molecule and the sample are processed together on the first solid support to improve the specific binding between the target molecule and the aptamer.

In the method of the present invention, when the target-aptamer complex is obtained in step (c), the competitor molecule and the sample are processed together on the first solid support to improve the specific binding between the target molecule and the aptamer.

In the method of the present invention, the isolating of the aptamer population from the complex population immobilized on the second solid support in step (g) in a form in which the aptamer population is immobilized on the first solid support, comprises: heating, treatment with a chaotropic salt, inducing a change of pH to strong acidity or strong basicity, treatment with a surfactant, or a combination thereof.

For other technical matters that are not specifically described above in describing the method of preparing an aptamer library specific to a target molecule population in a sample according to the present invention, the description about the method of generating a profile for a target molecule population in a sample according to the present invention or the description about the method of distinguishing two or more samples according to the present invention is valid as they are, and the descriptions may be referred to.

### Advantageous Effects

As described above, according to the present invention, a method of obtaining a profile of a target molecule population in a sample including an identified target molecule, using an aptamer is provided. In the method of the present invention, the target molecule population in the sample may be provided as an aptamer profile including an unknown target molecule, and this aptamer profile can be used to determine whether drug prescription is appropriate (i.e., anticancer drug companion diagnosis, etc.), to provide disease diagnosis information, to monitor drug treatment to determine drug compliance, to determine the degree or absence/presence of in vitro cellular response to drug treatment and to obtain useful information to humans for classification or identification of species, etc.

### BRIEF DESCRIPTION OF DRAWINGS

FIGS. 1, 2, 3, 4, and 5 are results showing that when using an RNA aptamer specific to a protein molecule population of the serum of a normal person, and using an aptamer library obtained for a protein molecule population in a serum sample of a lung cancer patient and an aptamer library obtained fora protein molecule population in a serum sample of a normal person, the two samples are distinguished.

FIGS. 6, 7, 8, 9, and 10 are results showing that when using an DNA aptamer specific to a protein molecule population of a serum, and using an aptamer library obtained for a protein molecule population in a serum sample of a lung cancer patient and an aptamer library obtained for a protein molecule population in a serum sample of a normal person, the two samples are distinguished.

### DETAILED DESCRIPTION

Hereinafter, the present invention will be described in detail with reference to some examples. However, the scope of the present invention is not limited to the examples

### <Example 1> Preparation of Reagent and Random Double-Stranded Nucleic Acid Library

### <Example 1-1> Reagent

HEPES, NaCl, KCl, EDTA, EGTA, MgCl₂, and Tween-20 were purchased from Fisher Scientific. Dextran sulfate sodium salt (DxSO4) was purchased from American International Chemical. KOD polymerase (KOD exo(-) polymerase) was purchased fromAvantor's VWR.

Tetramethylammonium chloride and 3-(cyclohexylamino)-2-hydroxy-1-propanesulfonic acid (CAPSO) were purchased from Sigma-Aldrich, and streptavidin phycoerythrin (SAP E) was purchased from Moss Inc. 4-(2-aminoethyl)-benzenesulfonylfluoride hydrochloride and 4-(2-aminoethyl)-benzenesulfonylfluoride hydrochloride (AEBSF) were purchased from Gold Biotechnology. Streptavidin coated 96-well plates (Pierce streptavidin coated plates HBC, clear 96 wells, product number 15500 or 15501) were purchased from Thermo Scientific. NHS-PEG4-Biotin (EZ-Link NHS-PEG4-Biotin, product number 21329) was purchased from Thermo Scientific, dissolved in anhydrous DMSO, and stored frozen in single-use aliquots. Yeast tRNA was purchased from Life Technologies.

PCR-grade natural and unnatural nucleotides were purchased from Thermo Fisher Scientific.

Random oligonucleotides, forward primers, reverse primers, and biotin-coupled forward primers were synthesized through order production by Baonia (in Korea).

SB18, which is a buffer solution, consists of 40 mM HEPES adjusted with NaOH to pH 7.5, 101 mM NaCl, 5 mM KCl, 5 mM MgCl2, and 0.05% by volume (vN) Tween 20. SB17, which is also a buffer solution, is obtained by adding 1 mM trisodium EDTA to SB18. SB17T, which is a buffer solution, consists of 40 mM HE PE S with pH 7.5, 102 mM NaCl, 5 mM KCl, 5 mM MgCl₂, 1 mM EDTA, and 0.05% Tween 20.

PB1, which is a buffer solution, consists of 10 mM HE PES adjusted to pH 7.5 with NaOH, 101 mM NaCl, 5 mM KCl, 5 mM MgCl₂, 1 mM trisodium EDTA, and 0.05% by volume (v/v) of Tween-20.

A CAPSO elution buffer solution consists of 100 mM CAPSO with pH 7.5 and 1 M NaCl. A neutralization buffer solution consists of 500 mM HEPES, 500 mM HCl and 0.05% by volume (v/v) of Tween-20.

As samples, serum collected at Asan Medical Center (IRB approval number 2015-0607) was used.

### <Example 1 2> Preparation of Random Double-Stranded Nucleic Acid Library

To prepare an RNA aptamer library that specifically binds to a target protein molecule population in the sample, first, single-stranded DNA oligonucleotides (random single-stranded nucleic acids) having the structure represented by General Formula I were prepared by Bioneer (Korea) by order.

<General Formula 1>

5′-CCACGCTGGGTGGGTCN₄₀GGACAAAGAGAGAAGAGAAAGAG-3′

The oligonucleotide having the structure represented by General Formula I consists of a 5 ̆conserved region, a variable region, and a 3 ̆conserved region arranged in this order.

In the above, the underlined nucleotide sequence is a conserved region that is a fixed region consisting of a known sequence, and N₄₀, which is a variable region, consists of 40 nucleotides, including bases such as A, G, T, C that occur with the same frequency.

A double-stranded DNA library was prepared by performing a PCR method using the oligonucleotide of General Formula I as a template. The primers used at this time were the following DS forward primer (SEQ ID NO: 2) containing the T7 promoter sequence and the following DS reverse primer (SEQ ID NO: 3):

<DS forward primer
(SEQ ID NO: 1)
5′-CCACGCTGGGTGGGTC-3′
<DS forward primer with T7 promoter sequence>
(SEQ ID NO: 2)
5′-TAATACGACTCACTATAGGGCCACGCTGGGTGGGTC-3′
<DS reverse primer
(SEQ ID NO: 3)
5′-CTCTTTCTCTTCTCTCTTTCTCC-3′

The DS forward primer containing the T7 promoter sequence (SEQ ID NO: 2) includes the T7 promoter sequence (the underlined portion in SEQ ID NO: 2) for RNA polymerase of bacteriophage T7 as well as the primer sequence for the 5′ conserved region (sequence of the underlined portion) of the single-stranded DNA oligonucleotide having the structure represented by General Formula I. The DS reverse primer (SEQ ID NO: 3) is a primer sequence for the 3′ conserved region (sequence of the underlined portion) of the single-stranded DNA oligonucleotide having the structure represented by General Formula I.

Polymerase chain reaction (PC R) was performed using the forward primer (SEQ ID NO: 2) and the reverse primer (SEQ ID NO: 3), with the single-stranded nucleic acid oligonucleotide of the structure of General Formula I used as a template.

Specifically, 1,000 pmoles of single-stranded nucleic acid oligonucleotides and 2,500 pmoles of primer pairs (DS forward primer containing a T7 promoter sequence and DS reverse primer) were mixed with 50 mM KCl, 10 mM Tris-Cl (pH 8.3), 3 mM MgCl2, 0.5 mM dNTP (dATP, dCTP, dGTP, and dTTP), and 0.1 U Taq DNA Polymerase (Perkin-Elmer, Foster City Calif.), for the PCR, and then purified with a QIAquick-spin PCR purification column (QIAGEN Inc., Chatsworth Calif.). In this way, double-stranded nucleic acid DNA having a T7 promoter was prepared. This PCR product was a double-stranded DNA containing a T7 promoter (sequence of the underlined portion), and the general structure thereof can be represented by General Formula II below.

<General Formula II>
5 [symbol]
TAATACGACTCACTATAGGGCCACGCTGGGTGGGTCN₄₀GGACAAAGAGA
GAAGAGAAAGAG-3′

### <Example 2> Preparation of Single-Stranded Nucleic Acid RNA Library

In this example, an RNA single-stranded nucleic acid library to be reacted with a protein molecule population in the sample was prepared. This RNA single-stranded nucleic acid library is a single-stranded nucleic acid library (SSN library) containing 2′-F-substituted pyrimidines which are modified nucleotides. It was synthesized by performing in vitro transcription of RNA single-stranded nucleic acids containing 2′-F-substituted pyrimidines, using the PCR product having the structure of general formula II, with the DuraScribe T7 Transcription Kit (EPICENTER, USA). After the synthesis, purification was performed.

Specifically, 200 pmoles of the prepared double-stranded DNA having the structure of General Formula II, 40 mM Tris-Cl (pH 8.0), 12 mM MgCl₂, 5 mM DTT, 1 mM spermidine, 0.002% Triton X-100, 4% PEG 8000, 5 U T7 RNA polymerase, and 1 mM ATP and GTP, and 3 mM 2′F-CTP and 2′F-UTP were mixed and reacted at 37 éC for 6 to 12 hours, followed by purification using a Bio-S pin 6 chromatography column (Bio-Rad Laboratories, Hercules Calif.). The amount and purity of the purified nucleic acid were determined with a UV spectrometer.

### <Example 3> Preparation of RNA Aptamer Library Specifically Binding to Protein Molecule Population of Sample, and Application Thereof to Sample Classification

### <Example 3-1> Preparation of SSN-FSS Library

(1) Preparation of SSN Library Tagged with First Tag

First all reagents and enzymes in Table 1 except for 30% PEG and DMSO were thawed on ice. The DMSO was thawed at room temperature, 30% PEG was warmed at 37 éC for 5 to 10 minutes until the volume becomes a liquid.

5 ≈ of the SSN library was transferred to a micro-centrifuge tube. After heating the SSN library at 85 éC for 3 to 5 minutes so that the SSN has a linear primary structure, the SSN library was immediately placed on ice.

Next, a biotin labeling reaction solution for the SSN library was prepared by adding reagents and enzymes in the order listed in Table 1 below.

TABLE 1
Reaction to ligate first tag to SSN library

| | Volume | Final Concentration | |
|---|---|---|---|
| Component | (≈L) | (Amount) | |
| Nuclease-free Water | 3 | — | |
| 10X RNA Ligase Reaction Buffer | 3 | 1X | |
| RNase Inhibitor | 1 | 1.33 U/≈L | (40 U) |
| SSN Library | 5 | 1.67 ≈M | (50 pmol) |
| Biotinylated Cytidine (Bis)phosphate | 1 | 33.3 ≈M | (1 nmol) |
| T4 RNA Ligase | 2 | 1.33 U/≈L | (40 U) |

| | | |
|---|---|---|
| 30% PEG | 15 | 15% |
| Total | 30 | — |

For the reaction solution, the last added reagent was 30% PEG. After carefully adding the 30% PEG to the reaction mixture with a pipette, the mixture was blended using a new pipette tip and reacted at 16 éC for 2 hours so that a reaction for ligating the biotin to the SSN was performed.

Next, 70 ≈ of nuclease-free water was added to the reaction solution, and 100 ≈ of chloroform:isoamyl alcohol was added thereto to extract RNA ligase. The reaction mixture was gently stirred and then centrifugated in a microcentrifuge at high speed for 2 to 3 minutes so that the phases were separated. Carefully, the upper aqueous layer containing biotinylated SSN was taken and transferred to a new tube.

In this way, a Bio-SSN library in which biotin as a first tag was added to the SSN 3 end of the SSN library was prepared.

The stock concentration of the prepared Bio-SSN library was 4 nM. The Bio-SSN library stock mix was diluted 4-fold with an SB17 buffer, heated at 95 éC for 5 minutes, and then cooled to 37 éC for 15 minutes before use. Streptavidin-coupled magnetic beads serving as a first support were washed twice with 150 mL of a P B1 buffer before use.

2) Preparation of SSN-FSS Library

133 ≈ of a suspension of streptavidin-coupled magnetic beads (Streptavidin-Coupled Dynabeads, Thermo Fisher Scientific, USA) serving as a first solid support (FSS) dissolved in 1×SB17, Tw (40 mM HEPES, 102 mM NaCl, 1 mM EDTA, 5 mM MgCl₂, 5 mM KCl, 0.05% Tween-20) was placed in a tube. An about 1.1× Bio-SS N library (with biotin added to the 3 end of the SSN) was thawed by vortexing. The 1.1× Biotin-SSN library was then boiled for 10 minutes, vortexed for 30 seconds, and cooled to 20 éC in a water bath for 20 minutes. Next, 100 ≈ of the cooled 1.1× Bio-SSN library was added to the tube in which the FSS (Streptavidin-Coupled Dynabeads) was contained. The mixture was protected from light and incubated at 25 éC for 20 minutes on a shaker set at 850 rpm so that the biotin portion of the Bio-SSN library and the avidin portion of the capture component of the FSS (Streptavidin-Coupled Dynabeads) were reacted to prepare a suspended SSN-FSS library.

The tube with the prepared suspended SSN-FSS library was centrifuged to remove the solvent 190 ≈ of a 1× CAPS prewash buffer (50 mM CAPS, 1 mM EDTA, 0.05% Tw-20, pH 11.0) was added, and the suspended SSN-FSS library was shaken for 1 minute. The CAPS prewash buffer was then removed by centrifugation. The CAPS washing and the centrifugation for removal of the CAPS buffer were then repeated once more.

For purification of the suspended SSN-FSS library, 190 ≈ of 1×SX17, Tw was added to the tube containing the suspended SSN-FSS library, and the suspended SSN-FSS library was then shaken for 1 minute. The 1×SB17, Tw was then removed by centrifugation. In addition, 190 ≈ of the 1×SX17, Tw was added, and the suspended SSN-FSS library was shaken for 1 minute. The 1×SB17, Tw was then removed by centrifugation (for 1 minute at 1000×g).

150 ≈ of a storage buffer (150 mM NaCl, 40 mM HEPES, 1 mM EDTA, 0.02% sodium azide, 0.05% Tween-20) was added to the tube containing the suspended SSN-FSS library. Thus, the SSN-FSS bead library in a suspension state was purified. The tube was carefully sealed and stored in darkness at 4 éC until use.

The solution to be used later was stored for a long time at −20 éC, and on the day of analysis, each suspended SSN-FSS library was thawed at 37 éC for 10 minutes, placed in a boiling water bath for 10 minutes, and cooled to 25 éC for 20 minutes before use.

After such heating and cooling, in actual use, 55 ul of each 2× suspended SSN-FSS library was manually pipetted into a 96-well PCR plate, and the tube was resealed with foil until use.

### <Example 3-2> Preparation of Analysis Sample

As samples, serums provided by Asan Medical Center (IRB approval number 2015-0607) were used.

To prevent protein degradation, each of the samples was diluted in 0.94×SB17 containing 0.6 mM MgCl₂, 1 mM Trisodium EDTA, 0.8 mM AEBSF, and yeast tRNA that is 5 times the SSN concentration. Thus, a 10% serum sample solution was prepared and used.

### <Example 3-3> Reaction Between SSN-FSS Library and Sample

55 ιL of the SSN-FSS library solution in a suspended state was added to a tube containing 55 ιL of the diluted sample solution. The sample composed of molecule populations and the suspended SSN-FSS library were mixed by pipetting, and the mixture was sealed with a foil cover. Thereafter, the tube was subjected to a reaction to form a complex (M-SSN-FSS) between the protein molecule (M) population of the sample and the SSN-FSS in a processing device at 37 éC for 3 hours and 30 minutes.

After the completion of the complex formation reaction, the tube containing 100 ιL of the reaction mixture was placed on a stand with a magnet, and the liquid layer was carefully removed with a pipette. The tube was washed 4 times with 300 ιL of a PB1 buffer containing 1 mM dextran sulfate and 50 ιM biotin (to block a first capture component). Then, the tube was washed 3 more times with 300 ιL of a PB1 buffer to prepare the M-SSN-FSS complex population.

### <Example 3-4> Second Tag Addition, Immobilization on Second Solid Support, and Elution of SS N-FSS Population

To the tube containing the M-SS N-FSS complex population, 150 ιL of a freshly prepared 1 mM NHS-PEG4-biotin solution in a PB1 buffer was added and treated with shaking for 5 minutes, and biotin serving as a second tag was added the molecules of the suspended M-SSN-FSS complexes.

The tube was centrifuged, the liquid was removed by suctioning, and the tube was washed 8 times with 300 ιl of a PB1 buffer supplemented with 10 mM glycerin. 100 ιl of a PB1 buffer supplemented with 1 mM dextran sulfate was added.

The buffer solution containing the second tag-added bio-M-SS N-FSS complex population in suspension in the tube was transferred to the wells of a plate coated with streptavidin serving as a second solid support (SSS). After allowing reaction at 800 rpm at room temperature for 10 minutes, the bio-M-SSN-FSS complex was induced to be immobilized on the second solid support through biotin serving as the second tag, so that an immobilized SSS-M-SSN-FSS complex was prepared.

Next, the liquid was carefully removed from the wells of the plate with a pipette, and the wells were washed 8 times with 300 ιL of a PB1 buffer supplemented with 25% propylene glycol (or 30% glycerol). The wells were washed 5 times with 300 ιL of a SB17T buffer, and finally the washing liquid was suctioned.

100 ιL of a CAPSO elution buffer was added, and the immobilized SSS-M-SSN-FSS complex was shaken for 5 minutes, so that the SS N-FSS complex was eluted. The SSN-FSS complex population was obtained by manually transferring 90 ιL of the solution from the wells of the plate to the wells of a PC R plate containing 10 ιL of a neutralization buffer.

### <Example 3-3> Preparation of RNA Aptamer Library Specifically Binding to Protein Molecule Population of Sample

F or the SSN-FSS complex population obtained above, RT-PCR and in vitro transcription were performed in the same manner as in Examples 1 and 2 above. Thus, finally, an RNA aptamer library that specifically binds to the protein molecule population of the sample was prepared.

An RNA aptamer library having higher specificity and affinity may be prepared by additionally performing the procedures of Examples 3-1 to 3-5 on the prepared RNA library at least once. However, since it is ideal that the RNA library specific to the protein molecule population in the sample should reflect the diversity of protein molecules included in the sample as it is, the steps of Examples 3-1 to 3-5 were performed only once, but the washing process was varied and repeated as described above to obtain an RNA aptamer library that well reflects the diversity of protein molecules included in the sample.

### <Example 3-6> Application to Differentiation Between Analysis Samples and Comparative Samples

The RNA aptamer library for the protein molecule population of each of the serum samples of normal persons obtained in Examples 3-5 was applied to classification of six serum samples (analysis samples L5, L7, L8, L10, L13, and L14) derived from lung cancer patients and six serum samples (comparative samples N1 to N6) derived from normal persons.

As test samples, lung cancer patient serum samples and normal person serum samples provided by Seoul Asan Hospital (IRB approval number 2015-0607) were used. In the analysis, the same procedures as in Examples 3-1 and 3-4 were performed on each sample to isolate an RNA aptamer population specifically binding to a protein molecule population in a test sample and to a protein molecule population in a control sample.

Specifically, after attaching the first tag to the RNA aptamer library, the first solid support (streptavidin) was reacted with magnetic beads to prepare an RNA aptamer library (SS N-FSS library) bound to the first solid support; and was then reacted with the sample to prepare a complex population (M-SSN-FSS complex population) bound to the protein population in the sample. Next the second tag was attached to the protein molecule of the complex, then reacted in the streptavidin (i.e., second solid support)-coated wells of a plate, and washed. Next; the aptamer population bound to the protein molecule population was eluted in a form (SSN-FSS complex form) bound to the first solid support.

Using the eluted complex population as a template and using the primers shown in Example 1 above, cDNA was prepared. Next, one-way PCR was performed on the cDNA once to obtain a collective double-stranded DNA fragment and an NGS library was prepared using Ion AmpliSeq Library Kit 20 (Ion Torrent, Thermo Fisher Scientific) according to the manufacturer's protocol. Next, with Ion Torrent (104, Thermo Fisher), the nucleotide sequence of each DNA fragment was analyzed. In addition, the occurrence frequency of each DNA fragment (i.e., the number or amount of the DNA fragments) and an aptamer profile, which is the overall information of the occurrence frequency of the DNA fragments, were obtained.

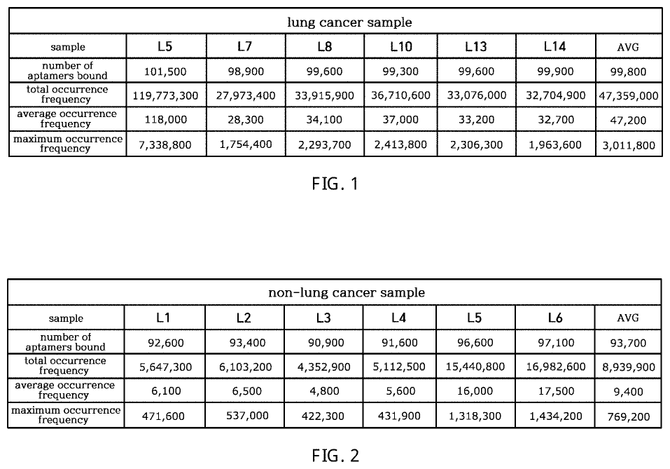

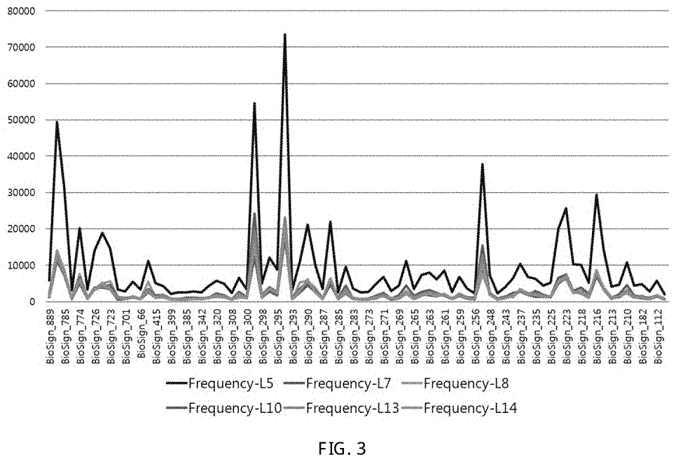

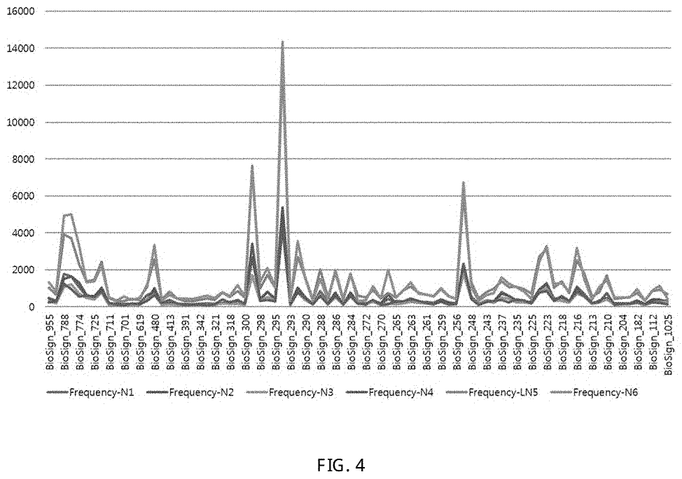

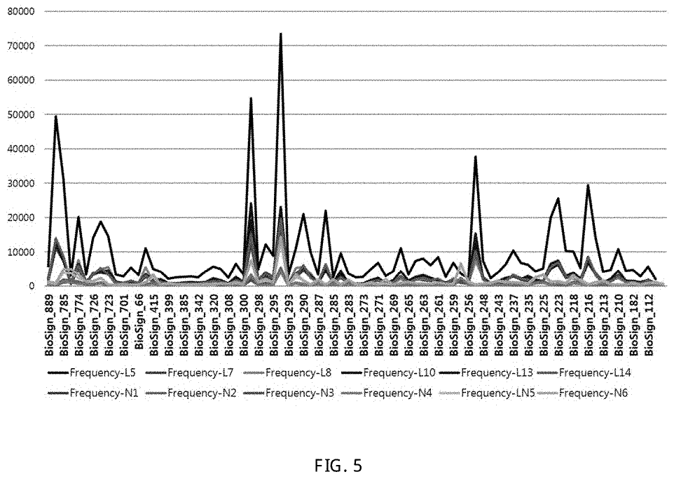

The results are shown in FIGS. 1 to 5. FIGS. 1 and 2 show the number of types of binding aptamers, the total occurrence frequency (i.e., total number of binding aptamers), the average frequency, and the maximum frequency in each analysis sample (lung cancer sample) and in each comparative sample (non-lung cancer sample), respectively. FIGS. 3 and 4 show aptamer profiles for some aptamers (BioSign_serial number) in the six lung cancer samples and aptamer profiles for some aptamers in the normal samples, respectively. FIG. 5 shows comparison between aptamer profiles for some aptamers that have a difference in the occurrence frequency in the lung cancer samples and the normal samples.

Referring to FIGS. 1 to 5, it is seen that the profile (the overall occurrence frequency of each RNA aptamer) of the RNA aptamer library reacted with the analysis target sample and the profile (the overall occurrence frequency of each RNA aptamer) of the DNA aptamer library reacted with the comparative sample are clearly distinguished.

### <Example 4> Preparation of DNA Aptamer Library Specifically Binding to Protein Molecule Population of Sample, and Application Thereof to Sample Classification

### <Example 4-1> Preparation of DNA Aptamer Library Specifically Binding to Protein Molecule Population of Sample

A forward primer (Bioneer, Korea) in which the oligonucleotide of <General Formula I> of Example 1 is used as a template and custom-made biotin is conjugated to the 5-end thereof and a reverse primer (Bioneer, Korea) in which biotin is not conjugated were used fora PC R reaction, so that biotin-attached double-stranded DNA was prepared. Here, it is necessary to check whether the amplified product is used or the oligonucleotide is used, as the template.

First, 10 ιM of an oligonucleotide template, 12 ιM of 5 ̆a biotin-conjugated primer, 0.5 mM of dNTP, and 0.25 U/mL of KOD polymerase (KOD exo(-) polymerase) were added to in 1×SQ20 buffer to perform a PCR reaction to prepare double-stranded DNA. Next the double-stranded DNA was captured on high-capacity MyOne-streptavidin paramagnetic beads (Invitrogen, catalog number 65001, Invitrogen, USA) serving as the first solid support (FSS) for 2 hours at room temperature, so that a double-stranded DNA-FSS complex library having a random sequence was prepared. It was progressed.

The prepared double-stranded DNA-FSS complex library was washed several times with a selected buffer (SBT: 40 mM HE PE S, pH 7.4; 102 mM NaCl; 5 mM KCl, 5 mM MgCl₂ 0.05% Tween 20). The complex library was suspended on 20 mM NaOH for 5 minutes to elute a modified sense strand. The eluent was neutralized to pH 7.4 with the required volume of 700 mM HCl, 180 mM HE PE S, pH 7.4, 0.45% Tween 20, and then concentrated with an Amicon Ultra-15 (Millipore) centrifugal filter to a residual volume of approximately 250 ≈.

In this way, a single-stranded DNA-FSS (‘SSN-FSS) complex library having a random sequence was prepared.

Next, the normal human serum sample prepared in Example 3-2 and 1000 or more (ℏ1000) pmol (˜10¹⁵) of the prepared SSN-FSS complex were mixed with SB17T buffer (40 mM HEPES, pH 7.5, 102 mM NaCl, 5 mM KCl, 5 mM MgCl₂, 1 mM EDTA, 0.05% Tween 20) to form an M-SSN-FSS complex, and were washed with SB17T buffer to remove unbound serum proteins, single-stranded DNA (SS N), and biotin complexes.

To the tube containing the M-SSN-FSS complex population, 150 ιL of a 1 mM NHS-PEG4-biotin solution freshly prepared in a PB1 buffer was added, and the tube was shaken for 5 minutes, so that biotin serving as a second tag was added to the molecules of the suspended M-SSN-FSS complexes. The addition of the biotin to the protein molecule can be achieved by covalent bonding between NHS-PEG4-biotin (Thermo Scientific, Pittsburgh, Pa., catalog no. 21329) and a lysine residue according to the manufacturer's protocol. Specifically, the M-SSN-The FSS complex population (300 pmol in 50 ≈) was exchanged with a Sephadex G-25 MicroSpin column provided with a SB17T buffer (40 mM HEPES, pH 7.5, 102 mM NaCl, 5 mM KCl, 5 mM MgCl₂, 1 mM EDTA, 0.05% Tween 20). The NHS-PE G4-biotin was added so as to become 30 WI, and the reactant was reacted at 4 éC for 16 hours to prepare a biotin-added complex, i.e., bio-M-SSN-FSS. Unreacted NHS-PEG4-biotin was removed with a Sephadex G-25 MicroSpin column.

The buffer solution containing the second tag-added bio-M-SS N-FSS complex population that was in a suspension state in the tube was transferred to the wells of a plate coated with streptavidin serving as a second solid support (SSS). After allowing reaction at 800 rpm at room temperature for 10 minutes, the bio-M-SSN-FSS complex was induced to be immobilized on the second solid support via the biotin serving as the second tag, so that an immobilized SSS-M-SSN-FSS complex was prepared.

Next, the liquid was carefully removed from the wells of the plate with a pipette, and the wells were washed 8 times with 300 ιL of a PB1 buffer supplemented with 25% propylene glycol (or 30% glycerol). The wells were washed 5 times with 300 ιL of a SB17T buffer, and finally the washing liquid was suctioned.

100 ιL of a CAPSO elution buffer was added, and the immobilize SSS-M-SSN-FSS complex was shaken for 5 minutes so that the SS N-FSS complex was eluted. The SSN-FSS complex population was obtained by manually transferring 90 ιL of the solution from the wells of the plate to the wells of a PC R plate containing 10 ιL of a neutralization buffer.

For the SSN-FSS population thus obtained, the PCR was performed in the same manner as in Examples 1 and 2 to prepare double-stranded DNA. As described above, a biotin-conjugated forward primer and a reverse primer were used to prepare biotin-conjugated double-stranded DNA. The biotin-conjugated double-stranded DNA was immobilized on the first solid support (FSS), and the sense strand was eluted as described above, thereby finally obtaining a DNA aptamer library having ability to specifically bind to the protein molecule population in the sample and being immobilized on the first solid support (FSS).

A DNA aptamer library with higher specificity and affinity may be prepared by repeating the above processes on the prepared DNA library one or more times. However, since it is ideal that the DNA library specific to the protein molecule population of the sample reflects the diversity of protein molecules contained in the sample, the above processes may be performed only once, but the washing process is diversified and repeated to obtain a DNA aptamer library that reflects well the diversity of protein molecules in the sample.

### <Example 4-2> Application to Differentiation Between Samples

The obtained DNA aptamer library immobilized on the first solid support (FSS) was used to distinguish six serum samples (analysis samples L5, L7, L8, L10, L13, and L14) derived from lung cancer patients and six serum samples (comparative samples N1 to N6) derived from normal persons.

As the samples, lung cancer patient serum samples and normal person serum samples provided by Seoul Asan Hospital (IRB approval number 2015-0607) were used. In the analysis, the same procedures as in Example 4-1 were performed on each sample to isolate DNA aptamer populations specifically binding to protein molecule populations in the test samples and the control samples in a state in which each of the aptamer populations was immobilized on the first solid support.

Specifically, the DNA aptamer library immobilized on the first solid support (FSS) was reacted with the sample to prepare an aptamer-protein complex population (C-SSN-FSS complex population). Next, a second tag was attached to the protein molecule of the complex, and then the complex was reacted in the wells of the plate coated with streptavidin serving as a second solid support, followed by washing. Next, the aptamer population coupled to the protein molecule population was eluted in a form coupled to the first solid support, i.e., an SSN-FSS complex form.

Using the eluted complex population as a template and using the primer suggested in Example 1, a collective double-stranded DNA fragment was obtained. An NGS library was prepared using Ion AmpliSeq Library Kit 20 (Ion Torrent, Thermo Fisher Scientific) according to the manufacturer's protocol. Next, the sequence of each DNA fragment was analyzed using Ion Torrent (104, Thermo Fisher), and the occurrence frequency of each DNA fragment (i.e., number or amount of DNA fragments) and an aptamer, which is comprehensive information on the occurrence frequency of each DNA fragment, were obtained.

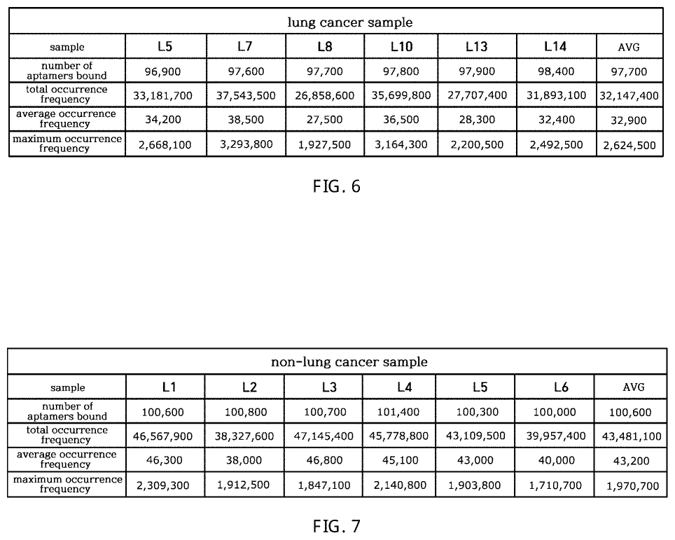

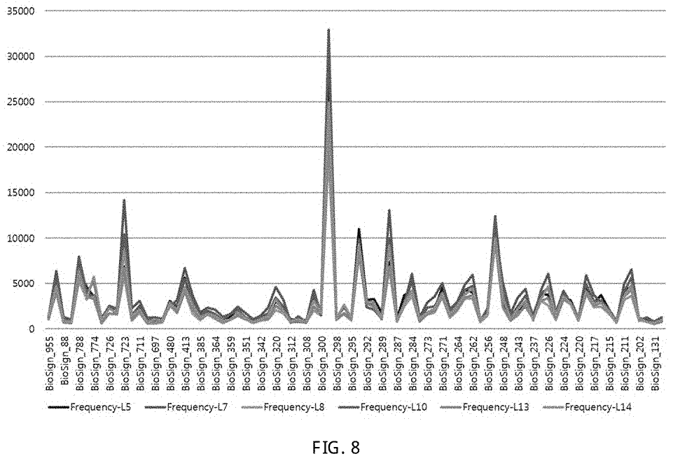

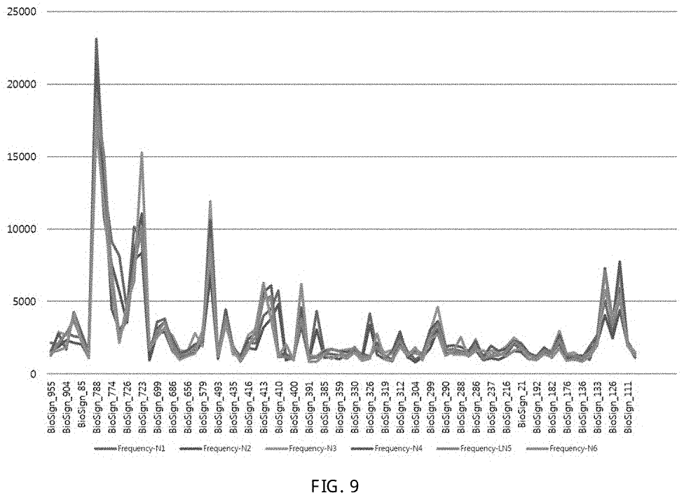

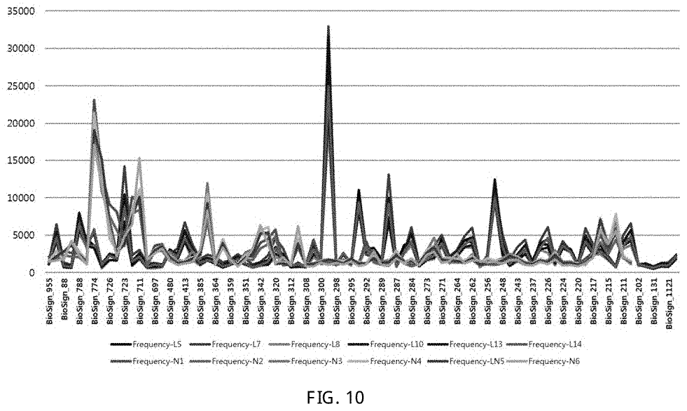

The results are shown in FIGS. 6 to 10. FIGS. 6 and 7 show the number of types of binding aptamers, the total occurrence frequency (i.e., total number of binding aptamers), the average frequency, and the maximum frequency in each analysis sample (lung cancer sample) and in each comparative sample (non-lung cancer sample), respectively. FIGS. 8 and 9 show aptamer profiles for some aptamers (BioSign_serial number) in six lung cancer samples and aptamer profiles for some aptamers in normal samples, respectively. FIG. 10 shows comparison between aptamer profiles for some aptamers that differ in the occurrence frequency in the lung cancer samples and the normal samples.

Referring to FIGS. 6 to 10, it can be seen that the profile (the overall frequency of appearance of each DNA aptamer) of the DNA aptamer library reacted with the analysis target sample and the profile (the overall frequency of appearance of each DNA aptamer) of the DNA aptamer library reacted with the comparative sample are clearly distinguished.

1. A method of generating a profile for a target molecule population in a sample, the method comprising:
(a) treating an aptamer library tagged with a first tag that is specific to a target molecule population in a sample and is capable of binding to a first capture component, with a solid support to which the first capture component is coupled so that the first capture component and the first tag are bound to each other, thereby immobilizing the aptamer library on the first solid support;
(b) treating an analysis target sample that is the same kind as the sample, with the first solid support on which the aptamer library is immobilized so that each target molecule of the target molecule population in the analysis target sample and each aptamer of the aptamer library form a target-aptamer complex, thereby obtaining a complex population;
(c) isolating the complex population in a state in which the complex population is immobilized on the first solid support by excluding unbound target molecules;
(d) attaching a second tag capable of binding to a second capture component to the target molecule of each complex of the isolated complex population;
(e) treating the complex population tagged with the second tag, with a second solid support to which the second capture component is coupled so that the second capture component and the second tag are bound and thus the complex population is immobilized on the second solid support;
(f) isolating an aptamer population from the complex population immobilized on the second solid support in a form in which the aptamer population remains immobilized on the first solid support; and
(g) generating an aptamer profile by quantifying each aptamer of the aptamer population that remains immobilized on the first solid support.

2. The method of claim 1, wherein the profile for the target molecule population in the sample is an aptamer profile.

3. The method of claim 1, wherein the aptamer profile is used to determine whether drug prescription is appropriate, provide disease diagnosis information, monitor drug treatment, determine drug adherence, determine whether there is a cellular response to drug treatment in vitro, determine a degree of a cellular response to drug treatment in vitro, to perform species classification, or identify a species.

4. The method of claim 1, wherein the target molecule is a protein, a glycoprotein, a lipoprotein, or a peptide.

5. The method of claim 1, wherein the target molecule population includes a target molecule that is unidentified.

6. The method of claim 1, wherein the aptamer library specific to the target molecule population in the sample is obtained by:
(i) preparing a single-stranded nucleic acid library having different random sequences, thereby having potential binding ability to various target molecules;
(ii) reacting the single-stranded nucleic acid library with the target molecule population in the sample to induce specific binding between each single-stranded nucleic acid and each target molecule to form a complex population;
(iii) isolating the complex population by excluding unbound single-stranded nucleic acids, and (iv) amplifying the single-stranded nucleic acids of the complex population.

7. The method of claim 1, wherein steps (i) to (iv) are performed once,
the excluding of the unbound single-stranded nucleic acids is performed through a washing step, and
the washing step is repeated two or more times, and the washing step is performed using a washing buffer containing a surfactant, a salt, a competitor molecule, or a mixture thereof.

8. The method of claim 1, wherein the aptamer is single-stranded RNA or single-stranded DNA.

9. The method of claim 8, wherein the single-stranded RNA or single-stranded DNA comprises nucleotides modified from sugar, phosphate, or base.

10. The method according to claim 1, wherein the sample is a biological sample or a processed sample thereof.

11. The method of claim 1, wherein the first tag and the second tag are biotin or an analog thereof, and the first capture component and the second capture component are streptavidin or an analog thereof.

12. The method of claim 1, wherein the first solid support is a magnetic bead and the second solid support is a non-magnetic support.

13. The method of claim 1, wherein when the target molecule and the aptamer form the complex in step (b), the competitor molecule and the sample are treated together on the first solid support to improve the specific binding between the target molecule and the aptamer.

14. The method of claim 1, wherein the isolating of the aptamer population from the complex population immobilized on the second solid support in step (f) in a form in which the aptamer population is immobilized on the first solid support comprises: heating, treatment with a chaotropic salt, inducing pH change to strong acidity or strong basicity, treatment with a surfactant, or a combination thereof.

15. The method of claim 1, wherein the quantification of the aptamer in step (g) is performed by next-generation sequencing technology, microarray method, or multiple real-time PCR method.

16.-46. (canceled)
